(19) United States
(12) Patent Application Publication
(10) Pub. No.: US 2020/0253860 A1
(43) Pub. Date: Aug. 13, 2020

(54) TOPICAL COMPOSITION FOR ANTIAGING BENEFITS

(71) Applicant: Conopco, Inc., d/b/a UNILEVER, Englewood Cliffs, NJ (US)

(72) Inventors: Paul Mark DIAS, Udupi (IN); Vijay Ramchandra GADGIL, Pune (IN); Nidhi JAGADISH, Bangalore (IN)

(73) Assignee: Conopco, Inc., d/b/a UNILEVER, Englewood Cliffs, NJ (US)

(21) Appl. No.: 16/638,084

(22) PCT Filed: Jul. 16, 2018
(86) PCT No.: PCT/EP2018/069236

§ 371 Date: Feb. 10, 2020

(30) Foreign Application Priority Data
Aug. 17, 2017 (EP) 17186643.7

(51) Int. Cl. A61K 8/9789; A61K 8/34; A61K 8/35; A61K 8/49; A61Q 19/08

(52) U.S. Cl. CPC A61K 8/9789; A61K 8/34; A61Q 19/08; A61K 8/498; A61K 8/35

(57) ABSTRACT

Disclosed is a topical composition comprising: (i) a first extract comprising botanical actives extracted from Emblica officinalis; and, (ii) a second extract comprising volatile botanical actives extracted from Camellia sinensis, wherein said second extract comprises E-2-hexenal and linalool at ratio of 0.1:1 to 10:1 parts by weight. Also disclosed is a method of activation of Nrf2, a method of upregulating gene expression of H01 in melanocytes and a method of upregulating gene expression of NQ01 in fibroblasts comprising a step of applying a topical cosmetic composition of the first aspect.

### FIELD OF THE INVENTION

The invention relates to a topical composition for activation of Nrf2 in certain human cells. Activation of Nrf2 is vital for antiaging benefits.

### BACKGROUND OF THE INVENTION

Human skin consists of two important layers, a thicker layer called dermis and a thinner layer on top of it called epidermis. The dermis is responsible for strength, elasticity and thickness. With aging, the thickness of this layer decreases and this is believed to be partially responsible for the appearance of wrinkles in aged skin. The epidermis is composed of a variety of cells which provide resilience and barrier properties. Keratinocytes account for 75 to 80% of the total population of cells in the epidermis. Within the epidermis, the keratinocytes are present in four distinct stages of differentiation. This differentiation is necessary for certain essential functions of the skin, namely for a protective barrier against environmental factors and to prevent loss of water from the body. The skin mainly consists of three main type of cells which are keratinocytes, melanocytes and fibroblasts.

Phase-2 enzymes are major detoxification enzymes and an important part of cellular defense against oxidants and other toxic chemicals.

While each individual Phase-2 gene may potentially be subjected to regulation through multiple mechanisms, it is the Keap1-Nrf2-ARE signaling system that is common to many Phase-2 genes. Nrf2, nuclear factor erythroid 2-related factor 2, is a transcription factor that is normally sequestered by its repressor Keap1. Binding with Keap1 promotes proteasomal degradation of Nrf2. Dissociation of Nrf2 from Keap1 allows the former to heterodimerize with partners, bind to a cis-acting DNA regulatory element, and promote transcription of the downstream gene. The DNA regulatory element to which Nrf2 heterodimer binds and activates is termed the antioxidant response element (ARE). One or more copies of the typical ARE sequence is known to exist in the 5′-flanking region of phase 2 genes including HO-1.

Each cell type, on its own, or in view of interaction with other types, plays a unique role in the appearance and health of skin. The nuclear factor erythroid 2-related factor 2 (Nrf2) is the master regulator of the skin's response to oxidative stress, which serves as a protective mechanism. Nrf2 regulates the downstream expression of various enzymes that regulate the antioxidant and detoxification mechanisms. Therefore, chemical agents that induce the activation of Nrf2 are universally used as screens to identify the actives that are suitable for various skin benefits, including inhibition of the synthesis of melanin in the melanocytes and the inhibition of reactive oxygen species in the fibroblasts and keratinocytes.

Herbal extracts are known for their detoxifying effects and some extracts are known to upregulate the expression of Nrf2. These days the potential of herbs can be determined by using antioxidant response element (ARE) cell line based high-throughput screening. Some herbs induce partial activation of Nrf2 which may be of the order of 2 to 3-fold increase.

WO14095198 A1 (Unilever) discloses the use of extracts of tea (Camellia sinensis) in cosmetic compositions for enhancement of immunity of the skin.

WO13060710 A2 (Unilever) discloses a topical cosmetic composition that contains extracts of green tea and extracts of black tea at selected ratio for anti-inflammation benefit.

U.S. Pat. No. 5,306,486 A (Unilever, 1994) discloses a cosmetic composition which includes green tea and a sunscreen compound. The composition blocks ultraviolet radiation.

US2005/0084566 A1 (Bavan) discloses a method of the production of instant tea soluble in hot water, comprising the steps of: (a) forming an extract by treating black tea leaves with hard warm water, (b) stripping the extract of its aroma volatiles by passing the tea extract through a flash evaporator under partial vacuum, (c) separating at least 12 wt % as insoluble solids from the extract by subjecting the extract to repeated clarification and polishing to obtain clarified concentrate (d) separating 6 to 10 percent soluble solids from the clarified concentrate, (e) adjusting pH of the concentrate to neutral by adding an edible acid, (f) adding aroma volatiles obtained in step (b) to the concentrate, and (g) obtaining a substantially moisture free tea powder capable of being reconstituted in hot water to produce instant tea that is substantially free of haze and cloudiness. In this process, the aroma is added back to the concentrate which is free of insoluble matter, and the method relates to production of hot water soluble instant tea, which is known to be substantially free of hot water insoluble tea solids.

US2007/0160737 A1 (Unilever) discloses a method of manufacturing an aromatic green tea product for consumption as a beverage, comprising the steps of providing an aroma composition comprising t-2-hexenal and linalool at weight ratio of at least 0.7:1 and combining the aroma composition with the tea product.

CN102091006 A (Long Run Tea Group, 2011) discloses a facial cleanser comprising 5 to 20 wt % tea extracts, 5 to 20 wt % coenzyme Q10, juice of Emblica offcinalis at 5 to 20 wt % and other things. The cleanser is said to provide ultraviolet resistance, anti-oxidation, astringe sweat pores, antibacterial, anti-acne, degreasing, radio resistance, anti-anaphylaxis, surplus fat removing, moisture preservation, aging resistance, smooth and elastic force improvement, wrinkle and microgroove prevention and free base removing; and the skin immunity is said to enhance after long term use, thus protecting the skin in a harmful environment.

US20080050459 A1 (3Lab Inc) discloses a cosmetic preparation comprising an effective amount of Phyllanthus emblica fruit extract and at least one oligopeptide such as Oligopeptide-4 and Oligopeptide-5 (pro-Elastin oligopeptide) The cosmetic preparation may further comprise Licorice extract, and Alpha-arbutin. The cosmetic preparation is meant for fine line and wrinkles and/or skin brightening.

Adhikari et.al. have disclosed in International Journal of Cosmetic Science, 2008, 30, 353-360, that methanol extracts of herbs, eg., E. officinalis and C. sinensis have the ability to inhibit activity of tyrosinase but the activity is rated at less than 50% of other herbs like Glycyrrhiza glabra and Morus alba.

The inhibitory effects of tannins contained in Phyllanthus emblica on melanin synthesis is discussed in Cosmetics & Toiletries 122 (2007), pp. 73-80 by Chaudhuri et.al.

### SUMMARY OF THE INVENTION

Botanical extracts of plants are widely used in cosmetic and pharmaceutical compositions. Often, they are used in combination, i.e., as separate or mixed extracts of two, three, four or even more plants. Most of the times, the extracts perform their own role, e.g., extract of the root of a plant X moisturizes the skin whereas extract of leaves of another plant Y increases the elasticity of skin. Very seldom are such extracts known to interact with each other to provide an altogether new effect or an effect which is more than the sum of their individual effects.

We have, under in vitro test conditions, unexpectedly observed a synergistic interaction between the botanical extracts of a first plant Emblica officinalis and a second plant Camellia sinensis. The extracts interact synergistically which manifests itself by activation of Nrf2 or its downstream genes in at least one of fibroblasts or melanocytes.

In accordance with a first aspect is disclosed a topical composition comprising:
(i) a first extract comprising botanical actives extracted from Emblica officinalis; and,
(ii) a second extract comprising volatile botanical actives extracted from Camellia sinensis,
   wherein said second extract comprises E-2-hexenal and linalool at ratio of 0.1:1 to 10:1 parts by weight.

In accordance with a second aspect is disclosed use of a topical composition of the first aspect for activation of Nrf2.

In accordance with a third aspect is disclosed use of a topical composition of the first aspect for upregulating gene expression of Heme Oxygenase-1 (HO-1) in melanocytes.

In accordance with a fourth aspect is disclosed use of a topical composition of the first aspect for upregulating gene expression of NQO1 in fibroblasts.

In accordance with a fifth aspect is disclosed a method of activation of Nrf2 comprising a step of applying a topical cosmetic composition of the first aspect.

In accordance with a sixth aspect is disclosed a method of upregulating gene expression of HO1 in melanocytes comprising a step of applying a topical composition of the first aspect.

In accordance with a seventh aspect is disclosed a method of upregulating gene expression of NQO1 in fibroblasts comprising a step of applying a topical composition of the first aspect.

In accordance with a further aspect is disclosed a topical composition of the first aspect for use to activate Nrf2.

In accordance with a further aspect is disclosed a topical composition of the first aspect for use to upregulate gene expression of HO1 in melanocytes.

In accordance with a further aspect is disclosed a topical composition for use to upregulate gene expression of NQO1 in fibroblasts.

In accordance with a further aspect is disclosed the use of a first extract comprising botanical actives extracted from Emblica officinalis and a second extract comprising volatile botanical actives extracted from Camellia sinensis in the manufacture of a topical composition for activation of Nrf2, wherein said second extract comprises E-2-hexenal and linalool at ratio of 0.1:1 to 10:1 parts by weight of the extract.

In accordance with a further aspect is disclosed the use of a first extract comprising botanical actives extracted from Emblica officinalis and a second extract comprising volatile botanical actives extracted from Camellia sinensis in the manufacture of a topical composition for upregulation of gene expression of HO1 in melanocytes, wherein said second extract comprises E-2-hexenal and linalool at ratio of 0.1:1 to 10:1 parts by weight of the extract.

In accordance with a further aspect is disclosed the use of a first extract comprising botanical actives extracted from Emblica officinalis and a second extract comprising volatile botanical actives extracted from Camellia sinensis in the manufacture of a topical composition for upregulation of gene expression of NQO1 in fibroblasts, wherein said second extract comprises E-2-hexenal and linalool at ratio of 0.1:1 to 10:1 parts by weight of the extract.

### DETAILED DESCRIPTION

These and other aspects, features and advantages will become apparent to those of ordinary skill in the art from a reading of the following detailed description and the appended claims.

Numerical ranges expressed in the format “from x to y” are understood to include x and y. When for a specific feature multiple preferred ranges are described in the format “from x to y”, it is understood that all ranges combining the different end points are also contemplated.

As used herein the term “comprising” encompasses the terms “consisting essentially of” and “consisting of”. Where the term “comprising” is used, the listed steps or options need not be exhaustive. Unless otherwise specified, numerical ranges expressed in the format “from x to y” are understood to include x and y. In specifying any range of values or amounts, any particular upper value or amount can be associated with any particular lower value or amount. Except in the examples and comparative experiments, or where otherwise explicitly indicated, all numbers are to be understood as modified by the word “about”. All percentages and ratios contained herein are calculated by weight unless otherwise indicated. As used herein, the indefinite article “a” or “an” and its corresponding definite article “the” means at least one, or one or more, unless specified otherwise. The various features of the present invention referred to in individual sections above apply, as appropriate, to other sections mutatis mutandis. Consequently, features specified in one section may be combined with features specified in other sections as appropriate. Section headings are added for convenience only, and are not intended to limit the disclosure in any way.

Emblica officinalis

Amla is an Ayurvedic herb. It is the fruit of the plant Emblica officinalis, also known as Phyllanthus emblica. The fruit is also known as Indian gooseberry. The plant, a member of Euphorbiaceae, grows to a medium-sized tree that is found in the plains and sub-mountain regions all over the Indian subcontinent. It is used in many edible herbal preparations as well as cosmetic compositions. The source and geographical origin of the extract of Emblica officinalis is detailed hereinafter.

It is preferred that the botanical actives from Emblica officinalis are extracted from its root, stem, leaves or bark by hydro-alcoholic extraction. It is preferred that first extract comprises more than 5% Ellagic acid by weight of the extract. In addition, the extract also comprises Vitamin C. Such an extract is commercially available with a vendor named Phyto Life Sciences Pvt Ltd, India having their website at http://www.plpl.in/herbal-extract-india.htm

The first extract is used as an ingredient in the compositions of the invention. It is preferred that the topical composition of the invention comprises botanical actives extracted from Emblica officinalis in an amount comprised in 0.001 to 10 wt % of said first extract.

Camellia sinensis

Compositions of the present invention comprise volatile botanical extracts from Camellia sinensis which is the tea plant. The plant is Camellia sinensis var. sinensis. Alternatively, it is Camellia sinensis var. assamica. Further alternatively, the volatile botanical extracts are obtained from combination the above two varieties.

It is preferred that the volatile botanical actives from Camellia sinensis are extracted from its leaves or fibres, or said actives are comprised in fractions obtained by vacuum stripping, steam distillation or fractionation of the volatile botanical actives.

A preferred process for preparation of volatile botanical actives from Camellia sinensis is as follows:

The process comprises the first step of squeezing out the juice from fresh leaves of the plant. This step leaves behind residue of squeezed leaves. The residue is dried in a dryer. The step leads to dried leaf residue and a dryer exhaust which is rich in aromatic compounds. The aromatic compounds are recovered from the exhaust as aroma condensate. Further details of the process can be found in US2007/0160737 A1 (Unilever).

Preferably the aroma condensate is distilled to concentrate it. This aroma condensate is the volatile botanical actives extracted from Camellia sinensis. The actives comprise E-2-hexenal and linalool at a ratio of 0.1:1 to 10:1 parts by weight.

It is preferred that the ratio is 0.1:1 to 5:1. In addition to the two ingredients named above, the extract preferably comprises other volatile ingredients, namely methyl salicylate. The volatile extracts of Camellia sinensis are distinguished from various other extracts reported elsewhere by the particular ratio of the two important ingredients present therein. It is preferred that the volatile botanical actives extracted from Camellia sinensis comprise more than 80 wt % volatile ingredients, more preferably more than 90 wt % and yet more preferably more than 95 wt % volatile ingredients. A further point of distinction is that extracts reported elsewhere, would usually mean extracts containing substantial amount of non-volatile ingredients. Some other prior art compositions containing extracts of Camellia sinensis contain volatile as well as non-volatile ingredients in which one or both the ingredients are missing or the ratio is completely different.

It is preferred that the compositions according to the invention comprise volatile botanical actives extracted from Camellia sinensis in an amount comprised in 0.001 to 10 wt % of said second extract.

### Further Details of the Composition of the Invention

“Topical composition” as used herein, is meant to include a composition for application to the external surface e.g. skin of mammals, especially humans for better skin health benefits. It is further preferred that the compositions of the invention is a cosmetic composition. Further preferably the compositions of the invention are leave-on or rinse-off compositions, preferably leave-on and includes any product applied to a human body primarily for enhanced health of skin but may be used also for improving the appearance, cleansing, odour control or general aesthetics. “Skin” as used herein is meant to include skin on the face and body (e.g., neck, chest, back, arms, underarms, hands, legs, buttocks and scalp) and especially to the under-eye portions of the face. The topical composition of the invention is especially useful for application on skin areas that get wrinkled or are more likely to get wrinkled especially to the sun exposed parts of the body.

The composition of the present invention could be in the form of a liquid, lotion, cream, foam, scrub, gel, soap bar or toner, or applied with an implement or via a face mask, pad or patch. Non-limiting examples of skin compositions include leave-on skin lotions and creams, shampoos, conditioners, shower gels, toilet bars, antiperspirants, deodorants, depilatories, lipsticks, foundations, mascara, sunless tanners and sunscreen lotions.

The compositions of the invention may be prepared according the usual manner of preparing such compositions. Reference may be made to standard text books and formulation guides.

When cosmetic composition is a cosmetic composition it preferably comprises a base preferably a dermatologically/cosmetically acceptable carrier. The carrier acts as a diluant, dispersant or carrier. The carrier may include materials commonly employed in skin compositions such as water, liquid or solid emollients, propellants, powders, emulsifiers, solvents, humectants, thickeners. The carrier in the compositions of the invention is single or mixtures of one or more vehicles. Preferably the carrier is present in the skin composition from 80 wt % to 99 wt %, preferably from 85 wt % to 90 wt %.

Compositions of the present invention will also include a cosmetically acceptable carrier. Water is the most preferred carrier. Amounts of water may, for example, range from 1 to 99%, preferably from 5 to 90%, more preferably from 35 to 70%, optimally between 40 and 60% by weight of the cosmetic composition. Ordinarily the compositions will be water and oil emulsions, which in some embodiments may be oil-in-water emulsions. Preferred emulsions are the water-in-oil variety.

Where the carrier is an emulsion, it is preferred that the particles are dispersed in the oil phase of the water and oil emulsion as this may improve the stability of the dye in the composition.

Emollient materials may be included as carriers in compositions of this invention. These may be in the form of silicone oils, synthetic esters and/or hydrocarbons. Amounts of the emollients may range, for example, anywhere from 0.1 to 95%, more preferably between 1 and 50% by weight of the composition.

Silicone oils may be divided into the volatile and nonvolatile variety. The term “volatile” as used herein refers to those materials which have a measurable vapor pressure at ambient temperature (25° C.). Volatile silicone oils are preferably chosen from cyclic (cyclomethicone) or linear polydimethylsiloxanes containing from 3 to 9, preferably from 4 to 5, silicon atoms. In many liquid versions of compositions according to the present invention, the volatile silicone oils may form a relatively large component of the compositions as carriers. Amounts may range, for example, from 5% to 80%, more preferably from 20% to 70% by weight of the composition.

Nonvolatile silicone oils useful as an emollient material include polyalkyl siloxanes, polyalkylaryl siloxanes and polyether siloxane copolymers. The essentially nonvolatile polyalkyl siloxanes useful herein include, for example, polydimethyl siloxanes. Among the preferred nonvolatile emollients useful in the present compositions are the polydimethyl siloxanes.

Organopolysiloxane crosspolymers can be usefully employed. Representative of these materials are dimethicone/vinyl dimethicone crosspolymers and dimethicone crosspolymers available from a variety of suppliers including Dow Corning (9040, 9041, 9045, 9506 and 9509), General Electric (SFE 839), Shin Etsu (KSG-15, 16 and 18 [dimethicone/phenyl vinyl dimethicone crosspolymer]), and Grant Industries (Gransil® brand of materials), and lauryl dimethicone/vinyl dimethicone crosspolymers, all trademarked materials supplied by Shin Etsu (e.g. KSG-31, KSG-32, KSG-41, KSG-42, KSG-43 and KSG-44). Amounts of the aforementioned silicone elastomers (when present) will usually be from 0.1 to 20% by weight dissolved usually in a volatile silicone oil such as cyclomethicone.

When silicones are present in large amounts as carrier and water is also present, the systems may be oil continuous. These normally will require emulsification with a water-in-oil emulsifier such as a dimethicone copolyol (e.g. Abil® EM-90 which is cetyl dimethicone copolyol).

Among the ester emollients are:

a) Alkenyl or alkyl esters of fatty acids having 10 to 20 carbon atoms. Examples thereof include isoarachidyl neopentanoate, isodecyl neopentanoate, isononyl isonanoate, cetyl ricinoleate, oleyl myristate, oleyl stearate, and oleyl oleate.
b) Ether-esters such as fatty acid esters of ethoxylated fatty alcohols.
c) Polyhydric alcohol esters. Butylene glycol, ethylene glycol mono and di-fatty acid esters, diethylene glycol mono- and di-fatty acid esters, polyethylene glycol (200 to 6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty esters, ethoxylated glyceryl mono-stearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters. Particularly useful are pentaerythritol, trimethylolpropane and neopentyl glycol esters of C1 to C30 alcohols. Exemplative is pentaerythrityl tetraethylhexanoate.
d) Wax esters such as beeswax, spermaceti wax and tribehenin wax.
e) Sterols esters, of which cholesterol fatty acid esters are examples thereof.
f) Sugar ester of fatty acids such as sucrose polybehenate and sucrose polycottonseedate.

Of particular use also are the C.sub.12-15 alkyl benzoate esters sold under the Finsolve® brand.

Hydrocarbons which are suitable cosmetically acceptable carriers include petrolatum, mineral oil, C.sub.11-C.sub.13 isoparaffins, polyalphaolefins, and especially isohexadecane, available commercially as Permethyl® 101A from Presperse Inc.

Humectants of the polyhydric alcohol-type can be employed as cosmetically acceptable carriers. Typical polyhydric alcohols include polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-butylene glycol, isoprene glycol, 1,2,6-hexanetriol, glycerol, ethoxylated glycerol, propoxylated glycerol and mixtures thereof. The amount of humectant may range, for example, anywhere from 0.5 to 50%, more preferably between 1 and 15% by weight of the composition. Most preferred is glycerol (also known as glycerin). Amounts of glycerin may range, for example, from 1% to 50%, more preferably from 10 to 35%, optimally from 15 to 30% by weight of the composition.

Besides cosmetically acceptable carriers, the compositions of this invention may include a variety of other functional ingredients. Sunscreen actives may be included in compositions of the present invention. These will be organic compounds having at least one chromophoric group absorbing within the ultraviolet ranging from 290 to 400 nm. Chromophoric organic sunscreen agents may be divided into the following categories (with specific examples) including: p-Aminobenzoic acid, its salts and its derivatives (ethyl, isobutyl, glyceryl esters; p-dimethylaminobenzoic acid); Anthranilates (o-aminobenzoates; methyl, menthyl, phenyl, benzyl, phenylethyl, linalyl, terpinyl, and cyclohexenyl esters); Salicylates (octyl, amyl, phenyl, benzyl, menthyl, glyceryl, and dipropyleneglycol esters); Cinnamic acid derivatives (menthyl and benzyl esters, alpha-phenyl cinnamonitrile; butyl cinnamoyl pyruvate); Dihydroxycinnamic acid derivatives (umbelliferone, methylumbelliferone, methylaceto-umbelliferone); Trihydroxycinnamic acid derivatives (esculetin, methylesculetin, daphnetin, and the glucosides, esculin and daphnin); Hydrocarbons (diphenylbutadiene, stilbene); Dibenzalacetone and benzalacetophenone; Naphtholsulfonates (sodium salts of 2-naphthol-3,6-disulfonic and of 2-naphthol-6,8-disulfonic acids); Dihydroxy-naphthoic acid and its salts; o- and p-Hydroxybiphenyldisulfonates; Coumarin derivatives (7-hydroxy, 7-methyl, 3-phenyl); Diazoles (2-acetyl-3-bromoindazole, phenyl benzoxazole, methyl naphthoxazole, various aryl benzothiazoles); Quinine salts (bisulfate, sulfate, chloride, oleate, and tannate); Quinoline derivatives (8-hydroxyquinoline salts, 2-phenylquinoline); Hydroxy- or methoxy-substituted benzophenones; Uric and vilouric acids; Tannic acid and its derivatives (e.g., hexaethylether); (Butyl carbityl) (6-propyl piperonyl) ether; Hydroquinone; Benzophenones (Oxybenzone, Sulisobenzone, Dioxybenzone, Benzoresorcinol, 2,2′,4,4′-Tetrahydroxybenzophenone, 2,2′-Dihydroxy-4,4′-d imethoxybenzophenone, Octabenzone; 4-Isopropyldibenzoylmethane; Butylmethoxydibenzoylmethane; Etocrylene; and 4-isopropyl-dibenzoylmethane). Particularly useful are: 2-ethylhexyl p-methoxycinnamate, 4,4′-t-butyl methoxydibenzoylmethane, 2-hydroxy-4-methoxybenzophenone, octyldimethyl p-aminobenzoic acid, digalloyltrioleate, 2,2-dihydroxy-4-methoxybenzophenone, ethyl 4-[bis(hydroxypropyl)]aminobenzoate, 2-ethylhexyl-2-cyano-3,3-diphenylacrylate, 2-ethylhexylsalicylate, glyceryl p-aminobenzoate, 3,3,5-trimethylcyclohexylsalicylate, methylanthranilate, p-dimethylaminobenzoic acid or aminobenzoate, 2-ethylhexyl p-dimethylaminobenzoate, 2-phenylbenzimidazole-5-sulfonic acid, 2-(p-dimethylaminophenyl)-5-sulfoniobenzoxazoic acid and mixtures thereof.

Particularly preferred are such materials as ethylhexyl p-methoxycinnamate, available as Parsol MCX®, Avobenzone, available as Parsol 1789®, Dermablock OS® (octylsalicylate) and Mexoryl SX® (with INCI name of Terephthalylidene Dicamphor Sulfonic Acid). Amounts of the organic sunscreen agent may range, for example, from 0.1 to 15%, more preferably from 0.5% to 10%, optimally from 1% to 8% by weight of the composition.

A variety of thickening agents may be included in the compositions. Illustrative but not limiting are stearic acid, Acrylamide/Sodium Acryloyldimethyltaurate Copolymer (Aristoflex AVC®), Hydroxyethyl Acrylate/Sodium Acryloyldimethyltaurate Copolymer, Aluminum Starch Octenyl Succinate, Polyacrylates (such as Carbomers including Carbopol® 980, Carbopol® 1342, Pemulen TR-2® and the Ultrez® thickeners), Polysaccharides (including xanthan gum, guar gum, pectin, carageenan and sclerotium gums), celluloses (including carboxymethyl cellulose, ethyl cellulose, hydroxyethyl cellulose and methyl hydroxymethyl cellulose), minerals (including talc, silica, alumina, mica and clays, the latter being represented by bentonites, hectorites and attapulgites), magnesium aluminum silicate and mixtures thereof. Amounts of the thickeners may range, for example, from 0.05 to 10%, more preferably from 0.3 to 2% by weight of the composition.

Preservatives can desirably be incorporated into the cosmetic compositions of this invention to protect against the growth of potentially harmful microorganisms. Suitable traditional preservatives for compositions of this invention are alkyl esters of para-hydroxybenzoic acid. Other preservatives which have more recently come into use include hydantoin derivatives, propionate salts, and a variety of quaternary ammonium compounds. Cosmetic chemists are familiar with appropriate preservatives and routinely choose them to satisfy the preservative challenge test and to provide product stability. Particularly preferred preservatives are phenoxyethanol, methyl paraben, propyl paraben, butyl paraben, isobutyl paraben, imidazolidinyl urea, sodium dehydroacetate and benzyl alcohol. The preservatives should be selected having regard for the use of the composition and possible incompatibilities between the preservatives and other ingredients in the composition. Preservatives are preferably employed in amounts ranging from 0.01% to 2% by weight of the composition.

Compositions of the present invention may also contain vitamins and flavonoids. Illustrative water-soluble vitamins are Niacinamide, Vitamin B2, Vitamin B6, Vitamin C and Biotin. Among the useful water-insoluble vitamins are Vitamin A (retinol), Vitamin A Palmitate, ascorbyl tetraisopalmitate, Vitamin E (tocopherol), Vitamin E Acetate and DL-panthenol. A particularly suitable Vitamin B6 derivative is Pyridoxine Palmitate. Among the preferred flavonoids are glucosyl hesperidin and rutin. Total amount of vitamins or flavonoids when present in compositions according to the present invention may range, for example, from 0.001 to 10%, more preferably from 0.01% to 1%, optimally from 0.1 to 0.5% by weight of the composition.

Desquamation agents are further optional components. Illustrative are the alpha-hydroxycarboxylic acids and beta-hydroxycarboxylic acids and salts of these acids. Among the former are salts of glycolic acid, lactic acid and malic acid. Salicylic acid is representative of the beta-hydroxycarboxylic acids. Amounts of these materials when present may range from 0.1 to 15% by weight of the composition.

A variety of herbal extracts may optionally be included in compositions of this invention. Illustrative are pomegranate, white birch (Betula Alba), green tea, chamomile, licorice, boswellia serrata, olive (Olea Europaea) leaf, arnica montana flower, Lavandula angustifolia, and extract combinations thereof. The extracts may either be water soluble or water-insoluble carried in a solvent which respectively is hydrophilic or hydrophobic. Water and ethanol are the preferred extract solvents.

Miscellaneous other adjunct cosmetic ingredients that may be suitable for the present compositions include ceramides (e.g. Ceramide 3 and Ceramide 6), conjugated linoleic acids, colorants (e.g. iron oxides), metal (manganese, copper and/or zinc) gluconates, allantoin, palmitoyl pentapeptide-3, amino acids (e.g. alanine, arginine, glycine, lysine, proline, serine, threonine, glumatic acid and mixtures thereof), trimethylglycine, sodium PCA, chelator like disodium EDTA, opacifiers like titanium dioxide, magnesium aspartate, and combinations thereof. Amounts may, for example, vary from 0.000001 to 3% by weight of the composition.

A small amount of emulsifying surfactant may be present. Surfactants may be anionic, nonionic, cationic, amphoteric and mixtures thereof. Levels may range, for example, from 0.1 to 5%, more preferably from 0.1 to 2%, optimally from 0.1 to 1% by weight. Advantageously the amount of surfactant present should not be sufficient for lather formation. In these instances, less than 2% by weight, preferably less than 1%, and optimally less than 0.5% by weight surfactant is present. Emulsifiers like PEG-100 stearate may be used as well as emulsion stabilizers like cetearyl alcohol and ceteareth-20 may be used and typically in amounts that do not exceed 5 percent by weight of the composition.

Other optional additives suitable for use in the composition of this invention include cationic ammonium compounds to enhance moisturization. Such compounds include salts of hydroxypropyltri (C1 to C3 alkyl) ammonium mono-substituted-saccharide, salts of hydroxypropyltri (C1 to C3 alkyl) ammonium mono-substituted polyols, dihydroxypropyltri (C1 to C3 alkyl) ammonium salts, dihydroxypropyldi (C1 to C3 alkyl) mono(hydroxyethyl) ammonium salts, guar hydroxypropyl trimonium salts, 2,3-dihydroxypropyl tri(C1 to C3 alkyl or hydroxalkyl) ammonium salts or mixtures thereof. In a most preferred embodiment and when desired, the cationic ammonium compound employed in this invention is the quaternary ammonium compound 1,2-dihydroxypropyltrimonium chloride. If used, such compounds typically make up from 0.01 to 30%, and more preferably from about 0.1 to about 15% by weight of the composition.

When cationic ammonium compounds are used, optional additives for use with the same are moisturizing agents such as substituted ureas like hydroxymethyl urea, hydroxyethyl urea, hydroxypropyl urea; bis(hydroxymethyl) urea; bis(hydroxyethyl) urea; bis(hydroxypropyl) urea; N,N′-dihydroxymethyl urea; N,N′-di-hydroxyethyl urea; N,N′-di-hydroxypropyl urea; N, N,N′-tri-hydroxyethyl urea; tetra(hydroxymethyl) urea; tetra(hydroxyethyl) urea; tetra(hydroxypropyl) urea; N-methyl-N′-hydroxyethyl urea; N-ethyl-N′-hydroxyethyl urea; N-hydroxypropyl-N′-hydroxyethyl urea and N,N′dimethyl-N-hydroxyethyl urea or mixtures thereof. Where the term hydroxypropyl appears, the meaning is generic for either 3-hydroxy-n-propyl, 2-hydroxy-n-propyl, 3-hydroxy-i-propyl or 2-hydroxy-i-propyl radicals. Most preferred is hydroxyethyl urea. The latter is available as a 50% aqueous liquid from the National Starch & Chemical Division of ICI under the trademark Hydrovance®. Such substituted ureas, while desirable in moisturizing formulations, are only selected for use when compatible with sunless tanning agent or agents (if present) used in the compositions of this invention.

Amounts of substituted urea, when used, in the composition of this invention range from 0.01 to 20%, more preferably from 0.5 to 15%, and most preferably from 2 to 10% based on total weight of the composition and including all ranges subsumed therein.

When cationic ammonium compound and substituted urea are used, in a most especially preferred embodiment at least from 0.01 to 25%, more preferably from 0.2 to 20%, and most preferably from 1 to 15% humectant, like glycerine, is used, based on total weight of the composition and including all ranges subsumed therein.

When making the compositions of this invention, ingredients are typically mixed with moderate shear under atmospheric conditions. Preferably, the compositions display a pH from 4 to 6.

Packaging for the composition of this invention can be a jar or tube as well as any other format typically seen for cosmetic, cream, washing and lotion type products. The compositions may be applied topically and preferably 1-4 milligrams of composition is applied per square centimeter of skin. The composition is preferably substantially white or colourless (transparent) when in packaged form but is transformable to a coloured composition on application to skin.

### Method and Use According to the Invention

In one aspect the use is non-therapeutic in nature. Alternatively, it is therapeutic in nature. When the use is non-therapeutic in nature, it preferably is for cosmetic purpose.

In one aspect the method of the invention is non-therapeutic in nature. Alternatively, it is therapeutic in nature. When the method is non-therapeutic in nature, it preferably is for cosmetic purpose.

The description of preferred aspects pertaining to the use of the composition applies mutatis mutandis to the method of the invention.

The method of the present invention may be carried out one or more times daily by applying to the skin which requires improvement epidermal skin barrier benefits. The amount of the composition used and the frequency with which it is applied may vary. In general, a small quantity of the composition, for example from 0.1 to 5 ml is applied to the skin. A rinsing step may optionally follow depending on whether the composition is formulated as a “leave-on” or a “rinse-off” product.

Now the invention will be demonstrated by means of following non-limiting examples.

### EXAMPLES

### Example 1: Nrf2 Activation in AREc32

The source of the hydro-alcoholic extract of the fruits of Emblica officinalis was Phyto LifeSciences P. Ltd. Ahmedabad, India (http://phytoherbs.in/product.html). It is commercially available with the vendor. The geographical origin of Emblica officinalis was India. Before use, the extract was diluted to 0.001% with the cell culture medium for fibroblasts referred below.

Volatile botanical extracts of Camellia sinensis were obtained in-house by following the method disclosed in US2007/0160737 A1 (Unilever). The geographical origin of the of the plant was India. The extract contained E-2-hexenal and linalool at a ratio of 0.14:1 parts by weight. Before use, the extract was diluted to 0.001% using the same cell culture medium as abovementioned.

The AREc32 cells were obtained from CRX biosciences (Dundee, Scotland, UK). AREc32 cells were seeded into 96-well plates (flat-bottom white, opaque, sterile, with lids) at a density of 2×10⁴, cells/well in 100 μL of complete media per well. Cell plates were then incubated at 37° C., 5% CO₂ in a 95% humidified incubator for 24 hours before active addition. Tert butylhydroquinone (TBHQ) was used as an assay positive control. For performing the Nrf2 activity assay, the cells were lysed using Passive Lysis Buffer (PLB) (5×) procured from Promega. The PLB was diluted in a 1:5 ratio with distilled water to make 1× PLB solution. 20 μL of 1× PLB was added to each well of the 96 well plate, including 2 wells without cell (control wells). The plate was covered with aluminium foil to protect from light and incubated for 20 minutes at room temperature, on a rocker incubator. After 20 minutes, 18 μL of the cell lysate was transferred from the transparent 96 well cell culture plate to an opaque, white, flat bottom 96-well plate. The white flat bottom plates were proceeded for Luciferase assay. The Luciferase assay was performed by using Luciferase Assay System, from Promega. 80 μL of the Luciferase Reagent was added using the injector on the TECAN infinite M1000 instrument. Luminescence was measured for a period of 10 seconds using the Green 1 filter. The typical delay time of 2 seconds, and read time of 10 seconds was used. 4 readings were taken from each well within 1 min. The plate was advanced to the next well for a repeat of the inject-then-read process. The luciferase value was represented as fold change over control in Table 1.

TABLE 1

| | | | | | 1 ml of medium |
|---|---|---|---|---|---|
| | | | Volatile | | containing 10 ppm of |
| | | | botanical extracts of | Botanical extracts of | Botanical actives of |
| | | | Camellia sinensis | Emblica officinalis | Camellia sinensis |
| | | | (1 ml of medium | (1 ml medium | and 500 ppm of |
| | | | containing 10 ppm | containing 500 ppm | botanical extracts of |
| Nrf2 | | TBHQ | of extract) | of the extract) | Emblica officinalis |
| activation | Control | 10 μM | in each well | in each well | in each well |
| Fold change | 1.0 | 4.5 | 10 | 4 | 28 |
| wrt control | | | | | |

### Note:

Tea leaves which were subjected to hypoxic withering (90 minutes) were also subjected to aqueous extraction and the extract was used in the experiments. It showed only 1.86-fold increase
Tea leaves which were subjected to non-hypoxic withering were also subjected to aqueous extraction and the extract was used in the experiments. It showed only 2.03-fold increase
Only Linalool showed barely any change wrt the control
Only E-2-Hexenal showed just 1.1-fold increase wrt the control
Linalool when combined with Botanical extracts of Emblica officinalis (1 ml medium containing 500 ppm of the extract) in each well showed no improvement over Emblica alone
E-2-Hexenal when combined with Botanical extracts of Emblica officinalis (1 ml medium containing 500 ppm of the extract) in each well showed no improvement over Emblica alone.

The data in Table 1 indicates that activation of Nrf2 is significantly more when the two extracts are together. The data further clearly indicates that the collective effect is synergistic because the effect is more than the predictable simple arithmetic addition of their individual effects and that too, at half the individual dosage.

### Example 2: HO1 Expression in Melanocytes

1*10\hat{ }5 HEMn (Human epidermal melanocytes neonatal) were plated in a 12-well plate and incubated at 37° C. in 5% CO₂ for 24 hours. Actives were added the next day and cells were harvested after 24 hours. Total cellular RNA was extracted from these cells using Ambion Purelink RNA mini Kit (CAT #12183020). The cDNA synthesis was carried out using Bio-Rad iScript (CAT #1708891). q PCR was then carried out using specific forward (AAGACTGCGTTCCTGCTCAAC) and reverse (AAAGCCCTACAGCAACTGTCG) primers for HO1. The PCR was carried out in Bio-Rad DNA engine gradient cycler system for 40 cycles in our case. The fold change was calculated using ΔΔcT method. (Reference WO2010/046316 A2, Unilever). The observations are tabulated in Table 2.

TABLE 2

| | | | | 1 ml of medium |
|---|---|---|---|---|
| | | Volatile | | containing 10 ppm of |
| | | botanical extracts of | Botanical extracts of | Botanical actives of |
| | | Camellia sinensis | Emblica officinalis | Camellia sinensis |
| | | (1 ml of medium | (1 ml medium | and 500 ppm of |
| Gene | | containing 10 ppm | containing 500 ppm | botanical extracts of |
| expression | | of the extract) | of the extract) | Emblica officinalis |
| of HO1 | Control | in each well | in each well | in each well |
| Fold change | 1.0 | 1.4 | 2.8 | 6.7 |
| wrt control | | | | |
| (β-actin) | | | | |

The data in Table 2 indicates that the gene expression of HO1 is significantly more when the two extracts are together. The data further clearly indicates that the collective effect is synergistic because the effect is more than the predictable simple arithmetic addition of their individual effects and that too, at half the individual dosage.

### Example 3: NQO1 Expression in Fibroblasts

1*10\hat{ }5 HDFa (Human dermal fibroblasts adult) were plated in a 12 well plate and incubated at 37° C. in 5% CO₂ for 24 hours. Actives were added the next day and cells were harvested after 24 hours. Total cellular RNA was extracted from these cells using Ambion Purelink RNA mini Kit (CAT #12183020). The cDNA synthesis was carried out using Bio-Rad iScript (CAT #1708891). q PCR was then carried out using specific forward (GAAGAGCACTGATCGTACTGGC) and reverse (GGATACTGAAAGTTCGCAGGG) primers for NQO1. The PCR was carried out in Bio-Rad DNA engine gradient cycler system for 40 cycles in our case. The fold change was calculated using ΔΔcT method. (Reference WO 2010/046316 A2, Unilever). The observations are tabulated in Table 3.

TABLE 3

| | | | | 1 ml of medium |
|---|---|---|---|---|
| | | Volatile | | containing 10 ppm of |
| | | botanical extracts of | Botanical extracts of | Botanical actives of |
| | | Camellia sinensis | Emblica officinalis | Camellia sinensis |
| | | (1 ml of medium | (1 ml medium | and 500 ppm of |
| Gene | | containing 10 ppm | containing 500 ppm | botanical extracts of |
| expression | | of the extract) | of the extract) | Emblica sinensis |
| of NQO1 | Control | in each well | in each well | in each well |
| Fold change | 1.0 | 2.0 | 4.5 | 8.0 |
| wrt control | | | | |
| (β-actin) | | | | |

The data in Table 3 indicates that the gene expression of NQO1 is significantly more when the two extracts are together. The data further clearly indicates that the collective effect is synergistic because the effect is more than the predictable simple arithmetic addition of their individual effects.

1. A topical composition comprising:
(i) a first extract comprising botanical actives extracted from Emblica officinalis; and,
(ii) a second extract comprising volatile botanical actives extracted from Camellia sinensis,
wherein said second extract comprises E-2-hexenal and linalool at ratio of 0.1:1 to 10:1 parts by weight.

2. A composition as claimed in claim 1 wherein said ratio is from 0.1:1 to 5:1 parts by weight.

3. A topical composition as claimed in claim 1 wherein said botanical actives from Emblica officinalis are extracted from its root, stem, leaves or bark by hydro-alcoholic extraction.

4. A topical composition as claimed claim 1 wherein said first extract comprises more than 5% Ellagic acid by weight of the extract.

5. A topical composition as claimed claim 1 wherein said volatile botanical actives from Camellia sinensis are extracted from its leaves or fibres, or said actives are comprised in fractions obtained by vacuum stripping, steam distillation or fractionation of said volatile botanical actives.

6. A topical composition as claimed in claim 1 comprising botanical actives extracted from Emblica officinalis in an amount comprised in 0.001 to 10 wt % of said first extract.

7. A topical composition as claimed in claim 1 comprising volatile botanical actives extracted from Camellia sinensis in an amount comprised in 0.001 to 10 wt % of said second extract.

8. Use of a topical composition as claimed in claim 1 for activation of Nrf2.

9. Use of a topical composition as claimed in claim 1 for upregulating gene expression of Heme Oxygenase-1 (HO-1) in melanocytes.

10. Use of a topical composition as claimed in claim 1 for upregulating gene expression of NQO1 in fibroblasts.

11. A method of activation of Nrf2 comprising a step of applying a topical cosmetic composition as claimed in claim 1.

12. A method of upregulating gene expression of HO1 in melanocytes comprising a step of applying a topical composition as claimed in claim 1.

13. A method of upregulating gene expression of NQO1 in fibroblasts comprising a step of applying a topical composition as claimed in claim 1.

14. A topical composition for use to activate Nrf2, comprising:
(i) a first extract comprising botanical actives extracted from Emblica officinalis; and,
(ii) a second extract comprising volatile botanical actives extracted from Camellia sinensis,
wherein said second extract comprises E-2-hexenal and linalool at ratio of 0.1:1 to 10:1 parts by weight.

15. A topical composition for use to upregulate gene expression of HO1 in melanocytes comprising:
(iii) a first extract comprising botanical actives extracted from Emblica officinalis; and,
(iv) a second extract comprising volatile botanical actives extracted from Camellia sinensis,
wherein said second extract comprises E-2-hexenal and linalool at ratio of 0.1:1 to 10:1 parts by weight of the extract.
